(19) United States
(12) Patent Application Publication
(10) Pub. No.: US 2022/0265533 A1
(43) Pub. Date: Aug. 25, 2022

(54) HYDRATING ORAL CARE COMPOSITIONS COMPRISING PEROXIDE

(71) Applicant: The Procter & Gamble Company, Cincinnati, OH (US)

(72) Inventors: Michael David Curtis, Mason, OH (US); Robert Scott Leonard, Fairfield, OH (US); Paul Albert Sagel, Maineville, OH (US); Jayanth Rajaiah, Loveland, OH (US)

(21) Appl. No.: 17/577,405

(22) Filed: Jan. 18, 2022

Related U.S. Application Data
(60) Provisional application No. 63/151,091, filed on Feb. 19, 2021.
(60) Provisional application No. 63/151,096, filed on Feb. 19, 2021.
(60) Provisional application No. 63/219,929, filed on Jul. 9, 2021.
(60) Provisional application No. 63/151,099, filed on Feb. 19, 2021.

(51) Int. Cl. A61K 8/22; A61K 8/25; A61K 8/26; A61K 8/19; A61K 8/21; A61K 8/44; A61K 8/06; A61K 8/31; A61Q 11/00

(52) U.S. Cl. CPC A61K 8/22; A61K 8/25; A61K 8/26; A61K 8/19; A61K 8/21; A61K 8/44; A61K 8/062; A61K 8/31; A61Q 11/00; A61K 2800/28

(57) ABSTRACT

Hydrating oral care compositions including peroxide and water. Hydrating dentifrice compositions including peroxide and water. Hydrating oral care compositions including peroxide and water that can release at least about 1% of the peroxide within 120 seconds. Hydrating oral care compositions including peroxide and water that can release at least about 15% of the peroxide within 15 minutes.

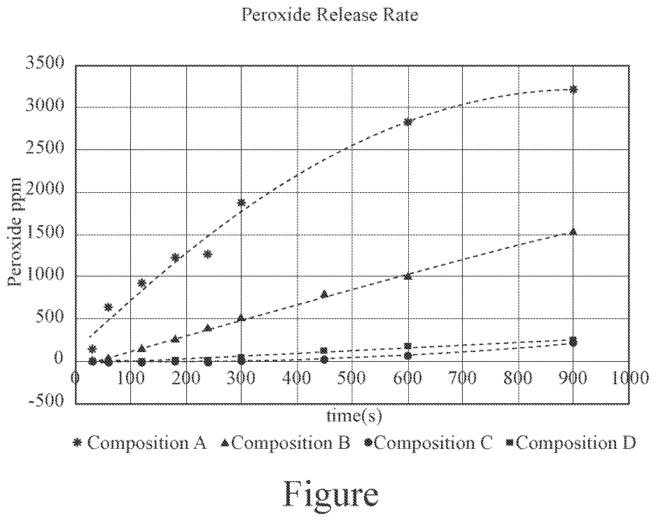

### FIELD OF THE INVENTION

The present invention relates to hydrating oral care compositions comprising peroxide and water. The present invention relates to hydrating dentifrice compositions comprising peroxide and water.

### BACKGROUND OF THE INVENTION

Oral care compositions, such as dentifrice compositions, can include fluoride, peroxide, and/or abrasive to clean teeth, prevent cavities, and maintain the aesthetics and health of the oral cavity, such as the teeth and gums.

It can be desirable to have dentifrice compositions with a relatively high level of peroxide, such as at least about 1%, at least about 3%, or at least about 3.5%, to achieve high whitening efficacy. However, it can be challenging to formulate dentifrice compositions with a relatively high level of peroxide because peroxide can be reactive with other dentifrice components, such as fluoride, metal ions, abrasives, etc. One approach to the formulation challenges associated with peroxide can be to separately package and/or apply peroxide from a separate whitening composition. However, consumer compliance can be lower when peroxide must be applied from a separate composition. Thus, it can be advantageous to provide peroxide in a dentifrice composition with other components, such as fluoride.

Another approach to the formulation challenges associated with peroxide can be to formulate peroxide as an adduct and/or complex in a nonaqueous chassis. However, anhydrous compositions can lead to the dehydration of oral care surfaces that can contribute to sensitivity, such as tooth sensitivity. Additionally, the complexation of peroxide can lead to lower amounts of available of peroxide to provide health and cosmetic benefits. As such, there is a need for an improved hydrating oral care composition, such as an improved hydrating dentifrice composition, including non-complexed peroxide.

### SUMMARY OF THE INVENTION

Disclosed herein is an oral care composition comprising (a) from about 1% to about 5%, by weight of the oral care composition, of peroxide; and (b) water, wherein the oral care composition releases at least about 1% of the peroxide within about 120 seconds according to the Peroxide Release Method.

Disclosed herein is a hydrating oral care composition comprising (a) from about 1% to about 5%, by weight of the oral care composition, of peroxide; and (b) water, wherein the oral care composition releases at least about 1% of the peroxide into an oral cavity of a user within about 120 seconds of initial application to the oral cavity.

Disclosed herein is an oral care composition comprising (a) from about 1% to about 5%, by weight of the oral care composition, of peroxide; and (b) water, wherein the oral care composition releases at least about 15% of the peroxide within about 15 minutes of initial application according to the Peroxide Release Method.

Disclosed herein is a hydrating oral care composition comprising (a) from about 1% to about 5%, by weight of the oral care composition, of peroxide; and (b) water, wherein the oral care composition releases at least about 15% of the peroxide into an oral cavity of a user within about 15 minutes of initial application to the oral cavity.

Disclosed herein is a jammed oil-in-water emulsion comprising (a) hydrophobic phase; (b) aqueous phase; and (c) peroxide, wherein the jammed oil-in-water emulsion releases at least about 1% of the peroxide within about 120 seconds according to the Peroxide Release Method.

Disclosed herein is a jammed oil-in-water emulsion comprising (a) hydrophobic phase; (b) aqueous phase; and (c) peroxide, wherein the jammed oil-in-water emulsion releases at least about 1% of the peroxide into an oral cavity of a user within about 120 seconds of initial application to the oral cavity.

Disclosed herein is an oral care composition comprising: (a) peroxide; (b) at least about 5%, by weight of the oral care composition, of water; and (c) fluoride and/or abrasive.

Disclosed herein is a method of delivering peroxide to an oral cavity of a user comprising (a) directing the user to apply any of the disclosed oral care compositions to the oral cavity; (b) directing the user to continue application of the oral care composition to the oral cavity for at least 120 seconds.

Disclosed herein is a method of delivering a majority of peroxide to an oral cavity of a user instead of a sink comprising (a) directing the user to a apply any of the disclosed oral care compositions to the oral cavity; (b) directing the user to continue application of the oral care composition to the oral cavity for at least 120 seconds; and (c) directing the user to expectorate the oral care composition.

### BRIEF DESCRIPTION OF THE FIGURE

The FIGURE shows the peroxide release over time for Composition A, B, C, and D.

### DETAILED DESCRIPTION OF THE INVENTION

The present invention is directed to hydrating oral care compositions comprising peroxide and water. While not wishing to being bound by theory, it is believed that peroxide that is stabilized through complexation and placed in an anhydrous chassis can lead to lower amounts of available of peroxide to provide health and cosmetic benefits. Additionally, anhydrous compositions can be dehydrating, leading to additional sensitivity challenges.

It has been surprisingly found that peroxide in composition including water can have a high peroxide release rate and hydrate the oral cavity. While not wishing to being bound by theory, it is believed that by minimizing reactivity with the remaining components in the oral care composition, including the abrasive, peroxide can be formulated in a composition including water.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival—paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen’ or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise peroxide and peroxide compatible abrasive, such as calcium pyrophosphate. Additionally, the oral care compositions can comprise other optional ingredients, as described below. The section headers below are provided for convenience only. In some cases, a compound can fall within one or more sections. For example, stannous fluoride can be a tin compound and/or a fluoride compound.

### Peroxide

The oral care composition comprises peroxide. The peroxide can include any suitable source of peroxide, such as solubilized peroxide compounds and/or solid peroxide sources. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, polyvinylpyrrolidone-peroxide complex, crosslinked polyvinylpyrrolidone-peroxide complex, and/or combinations thereof.

While not wishing to being bound by theory, it is believed that peroxide compounds that are stabilized through the complexation with a stabilizing compound, such as crosslinked polyvinyl pyrrolidone-peroxide, are less available to provide an oral health benefit, such as a whitening benefit, when applied to the oral cavity. Thus, the peroxide can comprise solubilized peroxide compounds, such as aqueous hydrogen peroxide.

Additionally, the oral care composition can be designed, such as through the selection of the other components of the oral care composition, to maximize the amount of remaining peroxide after a defined period after initially mixing the oral care composition. Suitable oral care compositions include oral care compositions comprising at least about 75%, at least about 80%, at least about 85%, at least about 90%, from about 90% to about 99%, or from about 88% to about 99% of hydrogen peroxide remaining in the oral care composition the oral care composition is stored at 50° C. for 10 days. Other suitable oral care compositions include oral care compositions comprising at least about 75%, at least about 80%, at least about 85%, at least about 90%, at least about 95%, from about 90% to about 99%, or from about 88% to about 99% of hydrogen peroxide remaining in the oral care composition the oral care composition is stored at 50° C. for 20 days.

The oral care composition can comprise from about 0.01% to about 20%, from about 0.01% to about 10%, from about 1% to about 5%, at least about 1%, at least about 2%, at least about 3%, at least about 3.5%, at least about 4%, or greater than 3%, by weight of the oral care composition, of the peroxide.

While not wishing to being bound by theory, it is believed that the peroxide, as described herein, can be un-bound, uncomplexed, and/or only minimally stabilized by other components in the oral care composition. It is additionally believed that oral care compositions comprising peroxide that is un-bound, uncomplexed, and/or only minimally stabilized to other components in the oral care composition, such as amphiphilic polymer and/or other thickening agents, can lead to a greater rate of release of peroxide than compositions that have peroxide adducts, such as polyvinylpyrrolidone-peroxide, and/or other sources of bound peroxide. Desirable oral care compositions include oral care compositions that have a peroxide release of at least about 10%, at least about 15%, at least about 25%, at least about 35%, at least about 50%, at least about 60%, at least about 70%, at least about 75%, at least about 80%, at least about 90%, at least about 95%, from about 10% to about 95%, from about 25% to about 75%, from about 20% to about 95%, from about 35% to about 90%, from about 40% to about 95%, from about 50% to about 90%, and from about 60% to about 99% in a defined period of time corresponding to a single oral care session, such as 30 second, 45 second, 60 seconds, 75 seconds, 90 seconds, 120 seconds, and/or 5 minutes.

### Abrasive

The oral care composition can comprise abrasive, such as a peroxide-compatible abrasive. Abrasives can be added to oral care formulations to help remove surface stains from teeth. The abrasive can comprise calcium abrasive, silica abrasive, carbonate abrasive, phosphate abrasive, alumina abrasive, other suitable abrasives, and/or combinations thereof.

The calcium abrasive can comprise calcium carbonate, dicalcium phosphate, tricalcium phosphate, calcium orthophosphate, calcium metaphosphate, calcium pyrophosphate, calcium polyphosphate, calcium hydroxyapatite, and combinations thereof.

The calcium abrasive can comprise calcium carbonate. The calcium-containing abrasive can be selected from the group consisting of fine ground natural chalk, ground calcium carbonate, precipitated calcium carbonate, and combinations thereof.

The calcium abrasive can comprise calcium pyrophosphate. While not wishing to be bound by theory, it is believed that particular sources of calcium pyrophosphate can be sufficiently compatible with peroxide to be used in an oral care composition comprising peroxide. Calcium pyrophosphate has at least three polymorphic phases: alpha (α), beta (β), and gamma (γ). It has been unexpectedly found that calcium pyrophosphate with a higher proportion of the γ phase are more compatible with peroxide, such as hydrogen peroxide. Suitable sources of calcium pyrophosphate can comprise a ratio of gamma phase calcium pyrophosphate to beta phase calcium pyrophosphate of at least about 0.75, at least about 0.8. at least about 0.9, at least about 1, from about 0.75 to about 1.5, from about 0.9 to about 1.5, or from about 1 to about 1.5.

While not wishing to being bound by theory, it is believed that abrasives that include high amounts of soluble metal ions are less compatible with peroxide due to reactivity between peroxide and metal ions. Thus, suitable sources of abrasives, such as calcium pyrophosphate, can comprise less than about 0.001%, less than about 0.00075%, less than about 0.0006%, less than about 0.0005%, or less than about 0.0001%, by weight of the abrasive. Additionally, the abrasive can be free of, substantially free of, or essentially free of soluble metal ions. Examples of trace metal ions that might be present in abrasives and have been previously reported to induce peroxide degradation include Cr, Mn, Fe, Co, Ni, Cu, Mo, and/or combinations thereof.

As described herein, it has also been unexpectedly found that abrasives with a slurry pH of from about 5.4 to about 7 were more compatible with peroxide. The slurry pH was determined by creating a 1:3 abrasive:water slurry and determining the pH.

The carbonate abrasive can comprise sodium carbonate, sodium bicarbonate, calcium carbonate, strontium carbonate, and/or combinations thereof.

The phosphate abrasive can comprise calcium phosphate, sodium hexametaphosphate, dicalcium phosphate, tricalcium phosphate, calcium orthophosphate, calcium metaphosphate, calcium polyphosphate, a polyphosphate, a pyrophosphate, and/or combinations thereof.

The silica abrasive can comprise fused silica, fumed silica, precipitated silica, hydrated silica, and/or combinations thereof.

The alumina abrasive can comprise polycrystalline alumina, calcined alumina, uncalcined alumina, fused alumina, levigated alumina, hydrated alumina, and/or combinations thereof.

Other suitable abrasives include diatomaceous earth, barium sulfate, wollastonite, perlite, polymethylmethacrylate particles, tospearl, and combinations thereof.

The oral care composition can comprise from about 0.01% to about 30%, from about 0.01% to about 15%, from about 0.01% to less than 15%, from about 1% to about 30%, from about 1% to less than 15%, from about 1% to about 12%, or from about 0.01% to about 10%, by weight of the oral care composition of the abrasive.

The oral care composition can have a Pellicle Cleaning Ratio (PCR) of at least about 75, at least about 90, at least about 100, from about 75 to about 250, or from about 100 to about 250.

The oral care composition can have a Relative Dentin Abrasion value of up to about 250, up to about 150, from about 70 to about 150, or from about 50 to about 250.

### Alumina

The oral care composition can include alumina as an abrasive. The alumina can be the sole abrasive or used in combination with other abrasives, as described herein. The abrasivity of alumina can be higher than silica, thus a lower amount of alumina abrasive can be added.

An oral care composition comprising alumina can have a hard tissue safety value (REA) of at least about 15, at least about 20, at least about 25, or at least about 40.

An oral care composition comprising alumina can have a pellicle cleaning ratio (PCR) of at least about 75, at least about 100, from about 75 to about 250, from about 50 to about 175, from about 50 to about 150, or rom about 65 to about 155.

An oral care composition comprising alumina can have a Relative Dentin Abrasion (RDA) of less than about 150, less than about 125, less than about 100, or less than about 90.

While not wishing to be bound by theory, it is believed that alumina can be reactive with peroxide and/or fluoride. Thus, desirable alumina abrasives include those that are compatible with peroxide and/or fluoride. Suitable oral care compositions include oral care compositions comprising at least about 75%, at least about 80%, at least about 85%, at least about 90%, at least about 95%, from about 90% to about 99%, or from about 88% to about 99% of hydrogen peroxide remaining in the oral care composition the oral care composition is stored at 40° C. for 30 and/or 60 days.

Other suitable oral care compositions include oral care compositions that comprise peroxide and alumina and have % loss of peroxide after 30 days and/or 90 days at 40° C. of less than about 10%, less than about 5%, less than about 4%, less than about 3%, from about 0.5% to about 10%, from about 0.5% to about 5%, or from about 0.1% to about 5%. While not wishing to being bound by theory, it is believed that alumina is unexpectedly stable with alumina, such that only a minimal amount of peroxide is lost to peroxide decomposition and/or degradation.

Other suitable oral care compositions include oral care compositions that comprise peroxide, fluoride, and alumina and have % loss of fluoride after 30 days and/or 90 days at 40° C. of less than about 20%, less than about 18%, less than about 15%, less than about 10%, less than about 5%, from about 0.5% to about 10%, from about 0.5% to about 20%, or from about 0.1% to about 15%. While not wishing to being bound by theory, it is believed that alumina and peroxide are unexpectedly stable with certain fluoride sources, such that only a minimal loss of fluoride is observed.

Other suitable oral care compositions include oral care compositions comprising peroxide and alumina and have a % increase of viscosity 30 days and/or 90 days at 40° C. of at least about 5%, at least about 10%, at least about 15%, at least about 20%, at least about 25%, at least about 35%, at least about 50%, at least about 75%, at least about 100%, from about 5% to about 125%, from about 5% to about 50%, or from about 1%, to about 75%. While not wishing to being bound by theory, it is believed that the combination of alumina and peroxide are unexpectedly stable, which can minimize the decrease in viscosity in meta-stable peroxide compositions normally observed due to the decomposition of peroxide.

The oral care composition can comprise from about 0.010% to about 10%, from about 0.010% to about 5%, from about 0.10% to about 5%, from about 0.010% to about 3%, or from about 0.010% to about 1%, by weight of the oral care composition, of alumina.

### Water

The oral care composition of the present invention can be a dentifrice composition that is anhydrous, a low water formulation, or a high water formulation. In total, the oral care composition can comprise from 0% to about 99%, from about 5% to about 60%, from about 5% to about 75%, about 20% or greater, about 30% or greater, about 50% or greater, up to about 45%, or up to about 75%, by weight of the composition, of water. Preferably, the water is USP water.

The oral care composition of the present invention can include water. While not wishing to be bound by theory, it is believed that including water in the oral care composition can lead to greater availability of peroxide to provide oral health benefits, such as whitening, while also hydrating the oral cavity. While one option to stabilize peroxide is to provide a complexed peroxide in an anhydrous dentifrice composition, it is believed that peroxide can be more effectively delivered through the selection of the other ingredients of the oral care composition while simultaneously hydrating the oral cavity.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 5% to about 45% or from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Amphiphilic Polymer

The oral care composition can comprise amphiphilic polymer. The amphiphilic polymer can be included as a peroxide compatible thickening agent. The amphiphilic polymer can comprise a polymer that has a hydrophobic portion and a hydrophilic portion. For example, the polymer can comprise a polymer macromolecule that comprises a hydrophilic backbone and hydrophobic subunits. This can allow the amphiphilic polymer to remain soluble in water while preventing strong interactions with the peroxide.

As described herein, it is believed that peroxide can be effectively delivered in an aqueous chassis if the other components are designed to minimize reactivity with the peroxide. As such, it is desirable for the amphiphilic polymer to not strongly react with the peroxide. Thus, it is desirable for the amphiphilic polymer to not form an isolatable complex with the peroxide.

The amphiphilic polymer can include polymers that are at least partially soluble and/or fully soluble in water.

Suitable amphiphilic polymers include 2-acrylamido-2-methylpropane sulfonic acid (AMPS) polymer, copolymer, cross-polymer, or combination thereof. In one example, the oral care composition can contain polyacrylate crosspolymer-6 (commercially available as SepiMAX™ ZEN from SEPPIC S.A., a subsidiary of the Air Liquide group, Puteaux Cedex, France).

The oral care composition can comprise from about 0.010% to about 10%, from about 0.10% to about 5%, from about 1% to about 10%, or from about 1% to about 5%, by weight of the oral care composition, of the amphiphilic polymer.

### Alkyl Alcohol

The oral care composition can comprise alkyl alcohol. While not wishing to be bound by theory, it is believed that the addition of alkyl alcohol in combination with the amphiphilic polymer can lead to oral care compositions with unexpectedly high viscosities.

The alkyl alcohol can include compounds with an alkyl functional group and an alcohol functional group. The alkyl functional can be linear, branched, cyclical, or combinations thereof. The alkyl alcohol can include a primary alcohol, a secondary alcohol, and/or a tertiary alcohol.

The alkyl alcohol can be represented by the general formula of CₙH₂ₙ₊₁OH, wherein n can be any whole number from 1 to 30, from 10 to 20, or from 10 to 25.

Specific examples of alkyl alcohols can include 1-heptacosanol, 1-hexacosanol, 1-nonacosanol, 1-octacosanol, 1-tetracosanol, docosanol, heneicosan-1-ol, pentacosan-1-ol, tricosan-1-ol, 1,4-butanediol, 1-heptanol, 1-hexanol, 1-nonanol, 1-octanol, 1-pentanol, 1-propanol, 2,4-dichlorobenzyl alcohol, 2-ethylhexanol, 3-nitrobenzyl alcohol, allyl alcohol, anisyl alcohol, arachidyl alcohol, benzyl alcohol, cetyl alcohol, cinnamyl alcohol, crotyl alcohol, furfuryl alcohol, isoamyl alcohol, neopentyl alcohol, nicotinyl alcohol, perillyl alcohol, phenethyl alcohol, propargyl alcohol, salicyl alcohol, stearyl alcohol, tryptophol, vanillyl alcohol, veratrole alcohol, and/or combinations thereof. The alkyl alcohol can comprise stearyl alcohol, cetyl alcohol, and/or combinations thereof.

The alkyl alcohol can be provided through commercial mixtures of alkyl alcohols, such as Lanette® W, which includes cetyl alcohol:stearyl alcohol:sodium lauryl sulfate at a 45:45:10 ratio (available from BASF, Florham Park, N.J.).

The oral care composition can comprise from about 0.010% to about 10%, from about 0.10% to about 5%, from about 1% to about 10%, or from about 1% to about 5%, by weight of the oral care composition, of the alkyl alcohol.

The combination of amphiphilic polymer and alkyl alcohol can lead to oral care compositions with unexpectedly high viscosities. Suitable ratios of amphiphilic polymer:alkyl alcohol can be at least about 0.25, at least about 0.5, at least about 0.75, at least about 1, from about 0.25 to about 5, about 0.5 to 1.5, or about 1.

The oral care composition can have a viscosity of at least about 5 cP, at least about 10 cP, at least about 20 cP, from about 5 cP to about 20 cP, or from about 1 cP to about 25 cP.

pH

The pH of the disclosed composition can be from about 4 to about 10, from about 4 to about 7, from about 4 to about 8, or from about 7 to about 10.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.10%, less than 0.010%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.010% to about 10%, from about 0.2% to about 10%, from about 0.4% to about 1%, or from about 0.3% to about 0.6%, by weight of the dentifrice composition. Alternatively, the oral care composition can be essentially free of, substantially free of, or free of zinc.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

### Orthophosphate

The oral care composition can comprise orthophosphate, which can be provided by an orthophosphate source. An orthophosphate source can comprise a salt including the orthophosphate anion, a salt including a phosphate anion (H₂PO₄⁻, HPO₄²⁻, and PO₄³⁻), a phosphoric acid compound, a polyphosphate source, which can breakdown into orthophosphate under a variety of conditions, and/or another suitable orthophosphate source.

The oral care composition can comprise from about 0.01% to about 15%, from about 0.1% to about 10%, from about 0.5% to about 5%, from about 1 to about 20%, or about 10% or less, by weight of the oral care composition, of the orthophosphate. Alternatively, the oral care composition can be essentially free of, substantially free of or free of orthophosphate.

### Surfactant

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

It was surprisingly found that additional amounts of anionic surfactant, such as alkyl sulfate surfactant and/or sodium lauryl sulfate, can lead to increased peroxide stability. As such, desirable compositions include compositions that comprise at least about 1.5%, greater than 1.5%, from about 1.5% to about 10%, or greater than 1.5% to about 10%, by weight of the oral care composition, of anionic surfactant.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula I, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

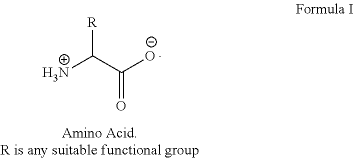

Suitable amino acids include one or more basic amino acids, one or more acidic amino acids, one or more neutral amino acids, or combinations thereof.

The oral care composition can comprise from about 0.010% to about 20%, from about 0.10% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Thickening Agents

The oral care composition can comprise one or more thickening agents in addition to the amphiphilic polymer and alkyl alcohol, as described herein. Thickening agents can be useful in the oral care compositions to provide a gelatinous structure that stabilizes the dentifrice and/or toothpaste against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether.

The thickening agent can comprise one or more inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.10% to about 10%, from about 0.2% to about 5%, or from about 0.5% to about 2% of one or more thickening agents.

### Dicarboxylic Acid

The oral care composition can comprise dicarboxylic acid. The dicarboxylic acid comprises a compound with two carboxylic acid functional groups. The dicarboxylic acid can comprise a compound or salt thereof defined by Formula II.

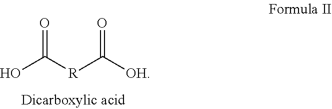

R can be null, alkyl, alkenyl, allyl, phenyl, benzyl, aliphatic, aromatic, polyethylene glycol, polymer, O, N, P, or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.01% to about 10%, from about 0.1% to about 15%, from about 1% to about 5%, or from about 0.0001 to about 25%, of dicarboxylic acid.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Hydrating Oral Care Composition

The oral care composition can be a hydrating oral care composition. Suitable hydrating oral care compositions can deliver a high amount of peroxide and/or other suitable active agent while hydrating or without removing water from the oral cavity upon expectoration of the oral care composition. Suitable oral care compositions can also include oral care compositions that can deliver a majority of the included active agent, such as peroxide, to the oral cavity, and not to the sink upon expectoration of the oral care composition to the sink.

Suitable oral care compositions include dentifrice compositions, toothpaste compositions, and/or jammed oil-in-water emulsions.

### Multi-Phase Oral Composition

The oral care composition can comprise multi-phase oral composition. The multi-phase oral composition can comprise multi-phase compositions, such as in U.S. Patent Application Publication Number 2018/0133121, U.S. Pat. Nos. 10,849,729, and/or 10,780,032, which are each herein incorporated by reference in their entirety.

Suitable multi-phase oral compositions include water-in-oil emulsions, oil-in-water emulsions, soluble particle dispersions in hydrophobic phase, jammed oil-in-water emulsions, and/or jammed water-in-oil emulsions. In particular, jammed oil-in-water emulsions, as described in TABLE 5 and TABLE 6, have a high peroxide release rate.

Traditional oil-in-water emulsions are multi-phase compositions with a discontinuous hydrophobic phase and a continuous aqueous phase. Stable oil-in-water emulsions can be prepared by combining a minority hydrophobic phase with a majority aqueous phase. Traditional oil-in-water emulsions are discontinuous droplets of hydrophobic phase suspended and/or stabilized within a continuous aqueous phase. As the hydrophobic and aqueous phases are immiscible, generally only a small portion of the hydrophobic phase can be stabilized within the aqueous phase before macroscopic separation occurs.

A high internal phase emulsion can be either oil-in-water or water-in-oil emulsion, wherein there is a high amount of the internal, discontinuous phase, by volume or weight of the multi-phase composition, relative to a traditional emulsion. A high internal phase emulsion can have more of the internal, discontinuous phase, by volume or weight of the total multi-phase composition than the external, continuous phase, by volume or weight of the multi-phase composition. However, the stability of high internal phase emulsions can prove challenging. High internal phase emulsions can suffer from macroscopic separation upon mixing or during storage of the high internal phase emulsions prior to use by a consumer.

As described herein, a jammed emulsion may be an unexpectedly stable high internal phase emulsion. As the concentration of the discontinuous phase of a high internal phase emulsion is increased, regions of discontinuous phase can become sufficiently crowded, such that they can jam against each other with a region of continuous phase between them and deform each other with a region of continuous phase between them. If both the continuous phase and discontinuous phase are liquids, the emulsion can transition into an at least a partially semisolid structure when the jamming transition occurs. The jammed emulsion, as described herein, can be prepared by the portion-wise addition or gradual addition or slow addition of the discontinuous phase to the continuous phase. Simply combining the entire discontinuous phase to the continuous phase will not necessarily result in jammed emulsion.

The multi-phase oral care composition and/or jammed oil-in-water emulsion can comprise hydrophobic phase, aqueous phase, active agent, such as peroxide, and optionally emulsifier.

### Aqueous Phase

The aqueous phase can be at least partially continuous, essentially continuous, or preferably continuous. The multi-phase oral care composition and/or jammed oil-in-water emulsion can comprise from about 0.01% to about 25%, from about 1% to about 20%, from about 2.5% to about 20%, or preferably from about 5% to about 15%, by weight or volume of the multi-phase oral care composition and/or jammed oil-in-water emulsion, of the aqueous phase.

The aqueous phase may also include other water-soluble solvents, such as for example, polyalkylene glycols with molecular weights from about 200 to about 20,000, humectants, or combinations thereof. Suitable humectants generally include edible polyhydric alcohols such as glycerin, sorbitol, xylitol, butylene glycol, and propylene glycol, and mixtures thereof. The aqueous phase may comprise at least about 10%, at least about 20%, or at least about 30%, of water, by weight or volume of the aqueous phase. The multi-phase oral compositions may comprise an aqueous solution of a bleaching agent, such as hydrogen peroxide, optionally including emulsifier.

### Hydrophobic Phase

The multi-phase oral care composition and/or jammed oil-in-water emulsion can comprise a hydrophobic phase. The hydrophobic phase can be at least partially discontinuous, essentially discontinuous, or preferably discontinuous.

The multi-phase oral care composition can comprise about 75% to about 99%, from about 80% to about 97.5%, greater than about 80%, greater than about 90%, or preferably, from about 85% to about 95%, by weight or volume of the multi-phase oral care composition or jammed oil-in-water emulsion, of the hydrophobic phase.

The density of the hydrophobic phase, as described herein, may be in the range of from about 0.8 g/cm³ to about 1.0 g/cm³, from about 0.85 g/cm³ to about 0.95 g/cm³, or about 0.9 g/cm³, or any other numerical range, which is narrower, and which falls within such broader numerical range, as if such narrower numerical ranges were all expressly written herein.

The hydrophobic phase can comprise a non-toxic oil, such as non-toxic edible oil. The hydrophobic phase can comprise non-toxic edible oils, saturated or unsaturated fatty alcohols, aliphatic hydrocarbons, long chain triglycerides, fatty esters, and combinations thereof. The hydrophobic phase may also comprise silicones, polysiloxanes, and mixtures thereof. The hydrophobic phase may be preferably selected from mineral oil, petrolatum, and combinations thereof.

### Emulsifier

The multi-phase oral care composition and/or jammed oil-in-water emulsion can comprise one or more emulsifiers. Depending on the design of the multi-phase oral care composition, the hydrophobic phase can have emulsifying properties. Thus, the emulsifier and the hydrophobic phase can comprise the same compound.

The multi-phase oral care composition and/or jammed oil-in-water emulsion, as described herein, can comprise from about 0.001% to about 20%, from about 0.01% to about 10%, up to about 10%, up to about 5%, or preferably from about 0.10% to about 10%, by weight of the multi-phase oral care composition or jammed oil-in-water emulsion, of the emulsifier.

Classes of surfactants useful as emulsifiers include nonionic, anionic, cationic, amphoteric, polymeric, synthetic emulsifiers, and mixtures thereof. Many suitable nonionic and amphoteric surfactants are disclosed by U.S. Pat. Nos. 3,988,433; 4,051,234, and many suitable nonionic surfactants are also disclosed by U.S. Pat. No. 3,959,458.

The emulsifier can comprise a polysorbate, an alkyl sulfate, Lipowax® D, or combinations thereof. Suitable polysorbate compounds include, polysorbate 20, 40, 60, 80, or combinations thereof, such as Tween® 20, 40, 60, 80, or combinations thereof.

The emulsifier can comprise natural emulsifiers, such as acacia, gelatin, lecithin and cholesterol; finely dispersed solids, such as colloidal clays, bentonite, veegum (magnesium aluminum silicate; and synthetic emulsifiers, such as salts of fatty acids, sulfates such as sorbitan trioleate, sorbitan tristearate, sucrose distearate, propylene glycol monostearate, glycerol monostearate, propylene glycol monolaurate, sorbitan monostearate, sorbitan monolaurate, poly oxy ethylene-4-lauryl ether, sodium lauryl sulfate, sulfonates such as dioctyl sosium sulfosuccinate, glyceryl esters, polyoxyethylene glycol esters and ethers, diethylene glycol monostearate, PEG 200 distearate, and sorbitan fatty acid esters, such as sorbitan monopalmitate, and their polyoxyethylene derivatives, polyoxyethylene glycol esters such as the monostearate, Polysorbate 80 (ethoxylated sorbitan monooleate) (supplied by Spectrum, etc.); and combinations thereof.

The emulsifier can be a surfactant that is non-reactive with a bleaching agent. For example, surfactants that are non-reactive with a bleaching agent may be substantially free of hydroxy groups, nitrogen groups and linkages, double or triple covalent bonds between adjacent carbon atoms, metals such as Zn, etc., or combinations thereof.

### Peroxide Release

Suitable oral care compositions include oral care compositions that have a high amount of peroxide release into the oral cavity. Suitable compositions include Compositions A and B, as described in TABLE 6. Other suitable compositions include oral care compositions that have a % peroxide diffusion of at least about 0.25%, at least about 0.5%, at least about 1%, at least about 1.5%, at least about 2.5%, at least about 5%, and/or at least about 10% after 2 minutes. Other suitable compositions include oral care compositions that have a % peroxide diffusion of greater than 13%, at least about 15%, at least about 25%, or at least about 45% after 15 minutes.

Other suitable compositions include oral care compositions that release at least about 0.25%, at least about 0.5%, at least about 1%, at least about 1.5%, at least about 2.5%, at least about 5%, and/or at least about 10% of peroxide into an oral cavity of a user within about 120 seconds of initial application to the oral cavity. Other suitable compositions include oral care compositions that release greater than 13%, at least about 15%, at least about 25%, or at least about 45% of peroxide into an oral cavity of a user within about 15 minutes of initial application to the oral cavity.

While not wishing to being bound by theory, it is believed that compositions with a high % peroxide diffusion according to the Peroxide Release Rate Method, as described herein, will also release a high amount of peroxide to the oral cavity.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

Peroxide Release

A 15 mL dialysis cell (2K MWCO, Slide-A-Lyzer G2 Dialysis Cassette) was filled with WFI MilliQ Water (16 grams) and the cap affixed. To one side of the cassette a test product was applied covering the entire cell membrane surface at a depth defined by the cell plastic housing by leveling the applied product with a spatula. A piece of parafilm was applied over the product composition to protect it during cassette mixing during sampling. The cell was placed either vertically or horizontally product facing down on a tared balanced. A timer was started post product application and samples of the WFI MilliQ water within the cassette were taken at the defined time points.

At each defined sample point, the cell was taken and inverted 180 degrees twice, the lid removed and a sample (0.3-0.50 g) pulled via a pipet. Following sampling, the dialysis cell was returned to the balance until the next sample was required. Each sample was assayed for percent peroxide as outlined below.

A Reflectoquant RQ Flex peroxide test strip reader (Millipore Sigma) was calibrated using both 0.2-0.20 mg/L (604) and 100-1000 mg/L (609) test strips (Supelco) with peroxide standard solutions as follows:

0.2-20.0 mg/L strips:

1. 5 grams of 35% Hydrogen Peroxide was diluted to 500 grams total with WFI MilliQ water.
2. 1 gram from (1) was diluted to 500 grams total weight using WFI MilliQ water
3. 2 drops from (2) were applied to a 0.2-20.0 mg/L test strip for 5 seconds and the excess solution dabbed on a paper towel.
4. The test strip was inserted into a RQ Flex 10 with the 0.2-20.0 mg/L test strip program loaded and the measurement recorded. Note total strip development and program is 15 seconds in duration.
5. 2 grams from solution (1) were diluted to 500 grams with WFI MilliQ water and steps (3) and (4) were repeated.

100-1000 mg/L strips:

1. 0.5 grams of 35% Hydrogen Peroxide was diluted to 500 grams total with USP water.
2. 2 drops from (1) were applied to a 0.2-20.0 mg/L test strip for 10 seconds and the excess solution dabbed on a paper towel. The strip was developed by sitting for an additional 50 seconds.
3. The test strip was inserted into a RQ Flex 10 with 10 seconds remaining on the 100-1000 mg/L test strip program and the measurement recorded. Note total strip development and program was approximately 60 seconds in duration for this test strip.
4. 1 gram of 35% Hydrogen Peroxide was diluted to 500 grams total with WFI MilliQ water
5. The test strip was inserted into a RQ Flex 10 with the 100-1000 mg/L test strip program loaded and the measurement recorded.

Sample Analysis

1. 2 drops from a given test sample time point were applied to a either a 0.2-20.0 mg/L or 100-1000 mg/L test strip following the development time period and analysis steps defined above (a3-a4 & b2-b3).
2. The diffused peroxide concentration defined the appropriate strip to use. If concentrations exceeded the respective strip concentration ranges, serial dilutions were performed to bring the concentration into range.

The peroxide release rate was determined for four compositions. Composition A, TABLE 1, and Composition B, TABLE 2A, are inventive compositions. Composition C and Composition D, TABLE 3, are commercial products marketed by the Colgate-Palmolive Company. Composition C is Colgate Optic White Renewal Toothpaste, which is marketed to include hydrogen peroxide at 3% wt %. Composition D is Colgate Optic White Overnight, which is also marketed to include hydrogen peroxide at 4 wt %.

TABLE 1
Composition A

| | Composition A |
|---|---|
| | (wt %) |
| Mineral Oil | 81.357 |
| Water | 14.141 |
| Hydrogen Peroxide | 3.0 |
| Sucralose | 0.35 |
| Polysorbate 20 | 1.0 |
| Flavor | 1.4 |
| Yield Stress | 12.70 Pa |

TABLE 1 shows the formulation for Composition A, a jammed oil-in-water emulsion. Composition A was produced by combining polysorbate 20, water, sucralose and the aqueous solution of H₂O₂ in a 20 gallon premix tank. The premix mixture was transferred to a 400 L vessel and agitated while mineral oil was slowly added over 30 minutes. Flavor was added next and the batch stirred for 5 minutes. An oil-in-water emulsion was formed during this step, and the composition developed a lotion-like, semisolid consistency.

TABLE 2A
Composition B

| | Composition B |
|---|---|
| | (wt %) |
| Water | 62.365 |
| Glycerin | 10.0 |
| Amphiphilic Polymer¹ | 1.375 |
| Sodium Lauryl Sulfate (28%) | 7.0 |
| Alkyl Alcohol¹ | 1.25 |
| Sodium Monofluorophosphate | 1.14 |
| Disodium Pyrophosphate | 0.7 |
| Tetrasodium Pyrophosphate | 0.3 |
| Sucralose | 0.33 |
| Calcium Pyrophosphate | 10.0 |
| H₂O₂ | 4.0 |
| Flavor | 1.54 |
| Total | 100 |

¹Sepimax Zen ™
²Cetyl Alcohol:Stearyl Alcohol:SLS (45:45:10)

TABLE 2A shows the formulation of Composition B, a dentifrice composition including fluoride, 4 wt % peroxide, water, and abrasive. The release of peroxide into the dialysis cell is shown in TABLE 5, and additionally shown in the FIGURE.

TABLE 2B
Compositions

| | Example | Example | Example | Example | Example | Example |
|---|---|---|---|---|---|---|
| | 1 | 2 | 3 | 4 | 5 | 6 |
| | (wt %) | (wt %) | (wt %) | (wt %) | (wt %) | (wt %) |
| Water | 56.33 | 56.83 | 57.33 | 56.58 | 56.83 | 57.08 |
| Glycerin | 10 | 10 | 10 | 10 | 10 | 10 |
| Amphiphilic Polymer¹ | 2.25 | 2.25 | 1.25 | 2.25 | 1.75 | 1.5 |
| SLS (28%) | 5 | 5 | 5 | 5 | 5 | 5 |
| Alkyl Alcohol² | 1.25 | 0.75 | 1.25 | 1 | 1.25 | 1.25 |
| Sodium Monofluorophosphate | 1.14 | 1.14 | 1.14 | 1.14 | 1.14 | 1.14 |
| Disodium Pyrophosphate | 0.7 | 0.7 | 0.7 | 0.7 | 0.7 | 0.7 |
| Tetrasodium Pyrophosphate | 0.4 | 0.4 | 0.4 | 0.4 | 0.4 | 0.4 |
| Sucralose | 0.3 | 0.3 | 0.3 | 0.3 | 0.3 | 0.3 |
| Calcium Pyrophosphate | 10 | 10 | 10 | 10 | 10 | 10 |
| H₂O₂ (35%) | 11.43 | 11.43 | 11.43 | 11.43 | 11.43 | 11.43 |
| Flavor | 1.2 | 1.2 | 1.2 | 1.2 | 1.2 | 1.2 |

TABLE 2B shows a variety of exemplary compositions that include different amounts of amphiphilic polymer (from 1.25 wt % to 2.25 wt %) and alkyl alcohol (from 0.75 wt % to 1.25 wt %). Ex. 1-6 include sodium monofluorophosphate (1.14 wt %), sodium lauryl sulfate (1.4 wt %), calcium pyrophosphate (10 wt %), and hydrogen peroxide (4 wt %).

TABLE 3
Comparative Compositions

| Composition C | Composition D |
|---|---|
| Colgate Optic White Renewal Toothpaste | Colgate Optic White Overnight |
| Propylene Glycol | Alcohol |
| Calcium Pyrophosphate | Acrylates/Octylacrylamide |
| PVP | Water |
| PEG/PPG-116/66 Copolymer | Hydrogen Peroxide |
| Hydrogen Peroxide | |
| Flavor | |
| Sodium Lauryl Sulfate | |
| Tetrasodium Pyrophosphate | |
| Sodium Saccharin | |
| Disodium Pyrophosphate | |
| Silica | |
| Sucralose | |
| BHT | |

TABLE 3 shows the ingredients listed on the carton for comparative examples and commercial products Composition C, Colgate Optic White Renewal toothpaste, and Composition D, Colgate Optic White Overnight whitening pen.

TABLE 4
Composition A, Peroxide Release Rate Sampling

| | | | | | | Adjusted | Avg |
|---|---|---|---|---|---|---|---|
| | | | Strip | Dilution | Readings | Readings | Reading |
| Sample | Time (s) | Weight (g) | (mg/L) | Factor | (mg/L) | (mg/L) | (mg/L) |
| 1 | 0 | 0.33 | 0.2-20 | NA | Non-Detect | NA | 0 |
| 2 | 30 | 0.34 | 100-1000 | NA | 163 | NA | 166 |
| | | | | | 169 | | |
| 3 | 60 | 0.44 | 100-1000 | NA | 402 | NA | 390 |
| | | | | | 377 | | |
| 4 | 120 | 0.37 | 100-1000 | NA | 701 | NA | 717 |
| | | | | | 733 | | |
| 5 | 180 | 0.42 | 100-1000 | 7.77 | 121 | 941 | 983 |
| | | | | | 132 | 1026 | |
| 6 | 240 | 0.41 | 100-1000 | 8.50 | 161 | 1368 | 1334 |
| | | | | | 153 | 1301 | |
| 7 | 300 | 0.37 | 100-1000 | 9.56 | 158 | 1511 | 1525 |
| | | | | | 161 | 1539 | |
| 8 | 450 | 0.46 | 100-1000 | 8.50 | 240 | 2040 | 2015 |
| | | | | | 234 | 1989 | |
| 9 | 600 | 0.49 | 100-1000 | 9.55 | 285 | 2722 | 2799 |
| | | | | | 301 | 2875 | |
| 10 | 900 | 0.50 | 100-1000 | 10.51 | 336 | 3531 | 3263 |
| | | | | | 285 | 2995 | |

TABLE 4 shows an example of the procedure utilized to calculate the amount of peroxide (ppm) in the dialysis cell. As shown in TABLE 4, Composition A led to 717 ppm of peroxide after 120 seconds, 1525 ppm of peroxide after 300 seconds, and 3263 ppm peroxide after 900 seconds. The same method of quantification shown in TABLE 4, was used to determine the concentration of the dialysis cell at various times with Composition B, Composition C, and Composition D.

TABLE 5
Peroxide Release
Peroxide Concentration (ppm) at Diffusion Time

| | Composition A1 | Composition B | Composition C | Composition D |
|---|---|---|---|---|
| Time | Inventive | Inventive | Comparative | Comparative |
| 0 | 0 | 0 | 0 | 0 |
| 30 | 166 | 15 | 0 | 0 |
| 60 | 390 | 39 | 0 | 0 |
| 120 | 717 | 150 | 0 | 2 |
| 180 | 983 | 268 | 1 | 10 |
| 240 | 1334 | 392 | 5 | 18 |
| 300 | 1525 | 513 | 9 | 45 |
| 450 | 2015 | 795 | 28 | 135 |
| 600 | 2799 | 1003 | 65 | 181 |
| 900 | 3263 | 1529 | 219 | 252 |

TABLE 5 shows the peroxide concentration of each dialysis cell after a loading of Composition A-D after a defined period of time. As shown in TABLE 5, Composition A and Composition B, resulted in 717 ppm and 150 ppm, respectively, of hydrogen peroxide in the dialysis cell after 120 seconds. Compositions C and D resulted in less than 1 ppm and 2 pm, respectively, of peroxide in the dialysis cell after 120 seconds. Additionally, Composition A and Composition B resulted in 3263 ppm and 1529 ppm, respectively, of peroxide in the dialysis cell after 900 seconds. Composition C and Composition D resulted in 219 ppm and 252 ppm, respectively, of peroxide in the dialysis cell after 900 seconds. Thus, membranes loaded with Compositions A and Composition B, both including hydrogen peroxide in solution, led to increased delivery of hydrogen peroxide across the membrane.

TABLE 6
% Peroxide Diffused after 2 Minutes

| | Product | Product | % Peroxide | % Peroxide |
|---|---|---|---|---|
| | Loading | % | Diffused after | Diffused after |
| Composition | (grams) | Peroxide | 2 minutes | 15 minutes |
| A | 3.55 | 3 | 10.8% | 49% |
| B | 3.99 | 4 | 1.5% | 15% |
| C | 4.61 | 3 | 0.001% | 2.5% |
| D | 0.76 | 4 | 0.13% | 13% |

TABLE 6 shows the relative amount of peroxide that diffused into the dialysis cell after a defined period of time. 9.3-10.8% of the total amount of peroxide in Compositions A diffused into the dialysis cell after 2 minutes. 49% of the total amount of peroxide in Composition A diffused into the dialysis cell after 15 minutes. 1.5% of the total amount of peroxide in Composition B diffused into the dialysis cell after 2 minutes. 16% of the total amount of peroxide in Composition B diffused into the dialysis cell after 15 minutes. In contrast, 0.001% and 0.13% of Composition C and Composition D diffused into the dialysis cell after 2 minutes and 2.5% and 13% of Composition C and Composition D diffused into the dialysis cell after 15 minutes.

Thus, compositions, such as toothpaste compositions and jammed oil-in-water emulsion compositions, that included peroxide and water led to an increased diffusion of peroxide into the dialysis cell. It has been surprisingly found that peroxide in composition including water can have a high peroxide release rate, as shown in TABLE 6 with Composition A and Composition B, and hydrate the oral cavity. While not wishing to being bound by theory, it is believed that by minimizing reactivity with the remaining components in the oral care composition, including the abrasive, peroxide can be formulated in a composition including water.

While not wishing to being bound by theory, it is believed that peroxide that is stabilized through complexation and placed in an anhydrous chassis can lead to lower amounts of available of peroxide to provide health and cosmetic benefits, as shown in TABLE 6 with Composition C and Composition D. Additionally, anhydrous compositions can be dehydrating, leading to additional sensitivity challenges.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hydrating oral care composition comprising:
(a) from about 1% to about 5%, by weight of the oral care composition, of peroxide; and
(b) water,
wherein the oral care composition releases at least about 1% of the peroxide within about 120 seconds according to the Peroxide Release Method.

2. The oral care composition of claim 1, wherein the peroxide comprises hydrogen peroxide, urea peroxide, carbamide peroxide, calcium peroxide, sodium peroxide, zinc peroxide, sodium percarbonate, polyvinylpyrrolidone-hydrogen peroxide complex, cross-linked polyvinyl pyrrolidone-hydrogen peroxide complex, or combinations thereof.

3. The oral care composition of claim 1, wherein the oral care composition comprises at least about 1%, by weight of the oral care composition, of the peroxide.

4. The oral care composition of claim 1, wherein the oral care composition comprises at least about 3%, by weight of the oral care composition, of the peroxide.

5. The oral care composition of claim 1, wherein the oral care composition comprises at least about 3.5%, by weight of the oral care composition, of the peroxide.

6. The oral care composition of claim 1, wherein the oral care composition comprises from about 5%, to about 75%, by weight of the oral care composition, of the water.

7. The oral care composition of claim 1, wherein the oral care composition comprises abrasive, the abrasive comprising silica, alumina, calcium abrasive, or combinations thereof.

8. The oral care composition of claim 7, wherein the calcium abrasive comprises calcium carbonate, calcium phosphate, calcium pyrophosphate, or combinations thereof.

9. The oral care composition of claim 7, wherein the alumina comprises calcined alumina, uncalcined alumina, or combinations thereof.

10. The oral care composition of claim 1, wherein the oral care composition is a dentifrice composition.

11. The oral care composition of claim 1, wherein the oral care composition comprises fluoride.

12. The oral care composition of claim 11, wherein the fluoride comprises sodium fluoride, stannous fluoride, sodium monofluorophosphate, amine fluoride, or combinations thereof.

13. The oral care composition of claim 11, wherein the fluoride comprises sodium monofluorophosphate.

14. The oral care composition of claim 1, wherein the oral care composition comprises metal.

15. The oral care composition of claim 14, wherein the metal comprises tin, zinc, copper, or combinations thereof.

16. The oral care composition of claim 15, wherein the tin comprises stannous chloride, stannous fluoride, or combinations thereof.

17. The oral care composition of claim 15, wherein the zinc comprises zinc lactate, zinc oxide, zinc phosphate, zinc citrate, zinc chloride, or combinations thereof.

18. The oral care composition of claim 1, wherein the oral care composition comprises amino acid.

19. The oral care composition of claim 18, wherein the amino acid comprises arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, or combinations thereof.

20. The oral care composition of claim 1, wherein the oral care composition comprises anionic surfactant.

21. The oral care composition of claim 20, wherein the oral care composition comprises at least about 1.5%, by weight of the oral care composition, of anionic surfactant.

22. The oral care composition of claim 20, wherein the oral care composition comprises greater than 1.5%, by weight of the oral care composition of anionic surfactant.

23. The oral care composition of claim 22, wherein the anionic surfactant comprises sodium alkyl sulfate surfactant.

24. The oral care composition of claim 23, wherein the sodium alkyl sulfate surfactant comprises sodium lauryl sulfate.

25. The oral care composition of claim 1, wherein the oral care composition comprises amphiphilic polymer.

26. The oral care composition of claim 25, wherein the amphiphilic polymer does not form an isolatable complex with the peroxide.

27. The oral care composition of claim 25, wherein the amphiphilic polymer comprises a macromolecule with a hydrophobic portion and a hydrophilic backbone.

28. The oral care composition of claim 25, wherein the amphiphilic polymer comprises a 2-acrylamido-2-methylpropane sulfonic acid polymer, copolymer, cross polymer, or combination thereof.

28. The oral care composition of claim 1, wherein the oral care composition comprises dentifrice composition, toothpaste composition, emulsion composition, or combinations thereof.

29. The oral care composition of claim 28, wherein the emulsion composition comprises oil-in-water emulsion, water-in-oil emulsion, or combinations thereof.

30. The oral care composition of claim 29, wherein the oil-in-water emulsion comprises jammed oil-in-water emulsion.

31. A jammed oil-in-water emulsion comprising:
(a) hydrophobic phase;
(b) aqueous phase; and
(c) peroxide,
wherein the jammed oil-in-water emulsion releases at least about 1% of the peroxide within about 120 seconds according to the Peroxide Release Method.

32. The jammed oil-in-water emulsion of claim 31, wherein the peroxide comprises hydrogen peroxide, urea peroxide, carbamide peroxide, calcium peroxide, sodium peroxide, zinc peroxide, sodium percarbonate, polyvinylpyrrolidone-hydrogen peroxide complex, cross-linked polyvinyl pyrrolidone-hydrogen peroxide complex, or combinations thereof.

33. The jammed oil-in-water emulsion of claim 31, wherein the jammed oil-in-water emulsion comprises at least about 1%, by weight of the jammed oil-in-water emulsion, of the peroxide.

34. The jammed oil-in-water emulsion of claim 31, wherein the jammed oil-in-water emulsion comprises at least about 3%, by weight of the jammed oil-in-water emulsion, of the peroxide.

35. The jammed oil-in-water emulsion of claim 31, wherein the hydrophobic phase comprises petrolatum.

36. The jammed oil-in-water emulsion of claim 31, wherein the jammed oil-in-water emulsion comprises emulsifier.

37. The jammed oil-in-water emulsion of claim 36, wherein the emulsifier comprises polysorbate, alkyl sulfate, or combinations thereof.

38. The jammed oil-in-water emulsion of claim 31, wherein the jammed oil-in-water emulsion comprises fluoride.

39. The jammed oil-in-water emulsion of claim 38, wherein the fluoride comprises sodium fluoride, sodium monofluorophosphate, amine fluoride, stannous fluoride, or combinations thereof.

40. The jammed oil-in-water emulsion of claim 31, wherein the jammed oil-in-water emulsion comprises metal.

41. The jammed oil-in-water emulsion of claim 40, wherein the metal comprises zinc, tin, copper, or combinations thereof.

42. The jammed oil-in-water emulsion of claim 41, wherein the zinc comprises zinc phosphate, zinc oxide, zinc citrate, zinc lactate, zinc chloride, or combinations thereof.

43. The jammed oil-in-water emulsion of claim 41, wherein the tin comprises stannous chloride, stannous fluoride, or combinations thereof.
